(19) United States
(12) Patent Application Publication
(10) Pub. No.: US 2017/0065190 A1
(43) Pub. Date: Mar. 9, 2017

(54) SENSOR FOR VENTRICULAR AND OUTFLOW TRACT OBSTRUCTION

(71) Applicant: Eric M. GREEN, San Mateo, CA (US)

(72) Inventor: Eric M. GREEN, San Mateo, CA (US)

(21) Appl. No.: 15/261,744

(22) Filed: Sep. 9, 2016

Related U.S. Application Data
(60) Provisional application No. 62/216,208, filed on Sep. 9, 2015.

(51) Int. Cl. A61B 5/0295; A61B 5/021; A61B 5/00; A61B 5/02; A61B 5/1455; A61B 5/026

(52) U.S. Cl. CPC A61B 5/0295; A61B 5/14552; A61B 5/0261; A61B 5/6824; A61B 5/6826; A61B 5/02028; A61B 5/021; A61B 5/7278; A61B 5/7282

(57) ABSTRACT

Systems and methods for identifying LVOT obstruction in a non-invasive manner are provided. The systems and methods include obtaining a plethysmographic signal from a patient, for example from a pulse oximeter. A waveform is generated from the signal. The signal is processed and analyzed to determine whether the patient suffers from LVOT obstruction.

### CROSS REFERENCE TO RELATED APPLICATIONS

This application claims benefit of U.S. Provisional Application No. 62/216,208 filed Sep. 9, 2015 which is herein incorporated by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Hypertrophic cardiomyopathy (“HCM”) is a condition characterized by hypercontractility and thickening of the heart muscle in the absence of another explanation such as hypertension or aortic stenosis. The most common symptoms in HCM patients are shortness of breath and chest pain with exertion. Among patients with HCM, the underlying cause for their symptoms can be difficult to determine. Symptoms may be attributed to impaired relaxation of the heart, inadequate blood flow to supply the thickened heart muscle or obstruction to the flow of blood out of the heart. This obstruction to the outflow of blood can be caused by a combination of the abnormal structure and function of the heart muscle and of the mitral valve. An obstruction may be fixed (present continuously) or dynamic (provoked), and it can be in the outflow tract or within the ventricular cavity. The identification of obstruction in HCM is important for both prognostic and therapeutic reasons. The presence of obstruction portends a worse prognosis but also suggests potential established medical therapies (beta blocker, calcium channel blocker, disopyramide) and interventional therapies (septal reduction). Existing methods for detection of ventricular and outflow tract obstruction include introduction of a pressure catheter into the left ventricle and echocardiographic assessment of blood flow and pressure gradients in the left ventricle and outflow tract. Both of these techniques are limited, particularly for dynamic obstruction, because they require trained operators, can only be performed episodically in the physician's office and may not reproduce the stimuli that provoke obstruction in an individual patient. This makes it difficult to correlate between study findings and a patient's symptoms during daily activities.

Cardiac obstruction such as that caused by hypertrophic cardiomyopathy can be fixed or can be dynamic in response to provocation, e.g., dehydration, a Valsalva maneuver, a ventricular premature beat or exercise, and it can change the flow of blood out of the heart in a manner detectable by analyzing arterial blood pressure measurements. For example, FIGS. 1A and 1B show blood pressure waveforms from a patient with HCM and dynamic obstruction measured at rest and during a Valsalva maneuver. The blood pressure measurements are from pressure catheters placed in the left ventricle (labeled “LV”) and in the femoral artery (labeled “FA”). While the LV blood pressure waveforms at rest and during Valsalva are nearly the same, the FA blood pressure waveforms in the two conditions are quite different with reduced amplitude and a characteristic “spike and dome” appearance during Valsalva. FIG. 2 shows the relationships among an ECG waveform, a left ventricular blood pressure waveform (LV, measured with a blood pressure catheter) and a femoral artery blood pressure waveform (FA, also measure with a blood pressure catheter) in a patient suffering from obstructive HCM. In FIG. 2, the first beat demonstrates a moderate pressure gradient demonstrated by the gap between the peaks in the LV and FA tracings. The second beat is a ventricular premature beat (VPB), as seen in the ECG tracing. As a result, the third (post-VPB) beat is more forceful and provokes greater outflow obstruction as demonstrated by the increased pressure gradient between the FA and LV tracings the “spike and dome appearance” and reduced pulse pressure of the FA tracing.

Information about the variation of blood pressure over time can also be obtained non-invasively. FIG. 3A illustrates an embodiment of a wrist sensor for detecting blood pressure. The sensor can be adhered to the skin, e.g., like the silicone tape sensor of FIG. 3A. Such a sensor can detect blood pressure from the radial artery using plethysmography. For example, non-invasive blood pressure sensors (such as, e.g., the NIBP100D noninvasive blood pressure system from Biopac Systems, Inc. or the SphygmoCor system from AtCor Medical) can be used to obtain quantitative blood pressure readings that can be used to generate a blood pressure waveform, as shown in FIG. 3B. Photoplethysmographic (PPG) information from optical sensors, such as pulse oximetry sensors or from plethysmography sensors in smart watches (e.g., Apple® watch or Samsung 1Z Gear® watch), can also be used to detect qualitative changes in arterial blood pressure over time to generate blood pressure waveforms, as shown in FIG. 4. In FIG. 4, the top two waveforms 42 and 44 show ECG information, the middle waveform shows a waveform 46 generated from signal taken from an arterial blood pressure sensor, and the bottom waveform 48 is a plethysmographic waveform from a pulse oximetry sensor.

### SUMMARY OF THE DISCLOSURE

One aspect of the current invention is a method of detecting hypertrophic cardiomyopathy with obstruction in a patient, the method comprising obtaining a plethysmographic signal from a patient; generating a waveform from the plethysmographic signal; and processing and analyzing the waveform to determine whether the patient suffers from hypertrophic cardiomyopathy with obstruction.

The plethysmographic signal can be a photoplethysmographic signal or a tonometric signal. In some embodiments, it is a pulse oximetry signal. Obtaining the plethysmographic signal can comprise obtaining a signal from a wrist or fingertip sensor. In some embodiments, processing and analyzing the waveform comprises estimating systolic time intervals (e.g., a left ventricular ejection time) from the waveform. Processing and analyzing the waveform can comprise determining a pressure gradient between a left ventricle and aorta of the patient. In some embodiments, processing and analyzing the waveform comprises estimating systolic time intervals (e.g., a left ventricular ejection time) from the waveform; and converting the left ventricular ejection time into a numerical pressure gradient between a left ventricle and an aorta of the patient. Obtaining the plethysmographic signal can comprise wirelessly obtaining the plethysmographic signal. In some embodiments, obtaining the plethysmographic signal occurs, at least in part, outside of a clinical setting. Obtaining the plethysmographic signal form the patient can comprise attaching a plethysmographic sensor to the patient. In some embodiments, the method further comprises displaying the result of processing and analyzing the waveform.

In another aspect of the invention a system for detecting hypertrophic cardiomyopathy with obstruction in a patient is provided. The system comprises a plethysmographic sensor; a processor in data communication with the plethysmographic sensor, the processor configured to generate a waveform from the information and to identify hypertrophic cardiomyopathy from the waveform; and an output mechanism configured to provide to a user the information from the blood sensor, the waveform and/or the indicator.

In some embodiments, the plethysmographic sensor is a pulse oximeter. The plethysmographic sensor can comprise a fingertip or wrist sensor. In some embodiments, the processor identifying hypertrophic cardiomyopathy comprises estimating a left ventricular ejection time from the waveform. In some embodiments, the processor identifying hypertrophic cardiomyopathy comprises determining a pressure gradient between a left ventricle and aorta of the patient.

In another aspect of the invention, a method for detecting a condition in a patient is provided. The method comprises obtaining a signal from a patient indicative of blood flow over time; generating a waveform from the signal; and processing and analyzing the waveform to determine whether the patient suffers from the condition.

In some embodiments, the signal is obtained from a photoplethysmographic sensor or a tonometry sensor. In some embodiments, processing and analyzing the waveform comprises estimating a left ventricular ejection time from the waveform; and converting the left ventricular ejection time into a numerical pressure gradient between a left ventricle and an aorta of the patient. Processing and analyzing the waveform can comprise determining a pressure gradient between a left ventricle and aorta of the patient. In some embodiments, the signal is generated from a wrist-worn or fingertip sensor. The condition can comprise at least one of HCM with obstruction, HCM without obstruction, dilated cardiomyopathy, restrictive cardiomyopathy, valvular heart disease, and heart failure with reduced or preserved ejection fraction.

### BRIEF DESCRIPTION OF THE DRAWINGS

FIGS. 1A and 1B illustrate blood pressure waveforms.

FIG. 2 illustrates an ECG waveform and blood pressure waveforms.

FIG. 3A illustrates an embodiment of a wrist sensor.

FIG. 3B illustrates a blood pressure waveform.

FIG. 4 illustrates blood pressure waveforms.

FIGS. 5A and 5B illustrate photoplethysmographic traces from a wristworn sensor.

FIG. 6 illustrates an embodiment of a diagnostic system.

FIG. 7 illustrates an embodiment of a method for identifying a condition in a patient.

FIG. 8 illustrates data from an experimental protocol.

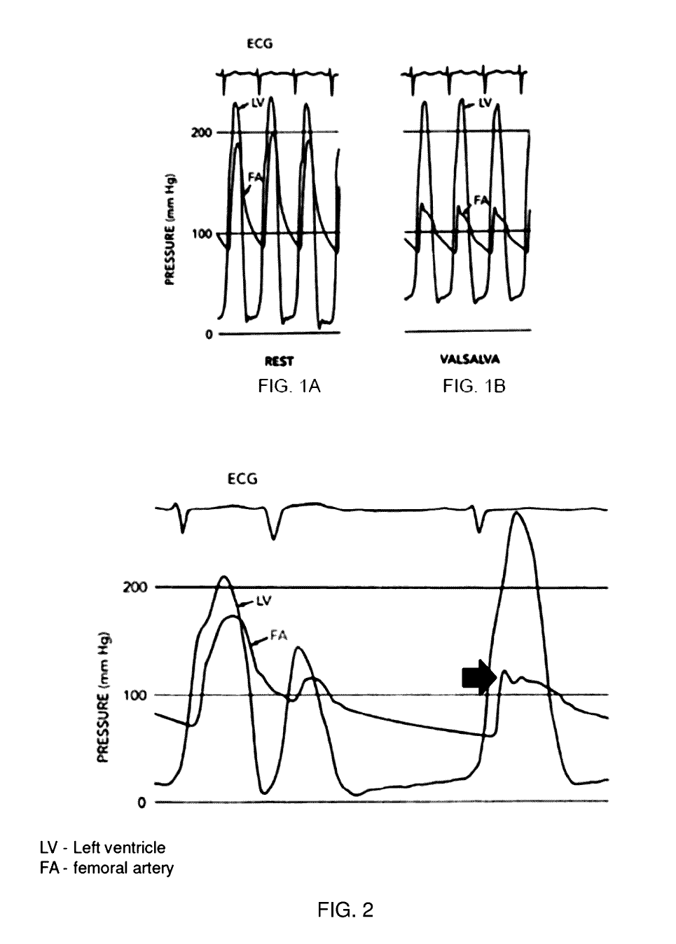

### DETAILED DESCRIPTION

As described above, the current state of the art for diagnosis of LVOT obstructions is episodic, involving introduction of a pressure catheter into the left ventricle or echocardiographic assessment of blood flow and pressure gradients in the left ventricle and outflow tract. In contrast, the present disclosure advantageously comprises non-invasive, continuous means for identifying and diagnosing LVOT obstruction. The inventors have found that blood flow data, including plethysmographic and tonometric data, can be used to identify heart conditions such as LVOT obstruction. Because of the non-invasive and convenient nature of these sensors, a physician can obtain data outside of a clinical setting, while a patient is going about his or her daily activities. This flexibility can allow for more accurate identification and diagnosis of a cardiac condition as the condition can manifest outside of the limited time the patient is in the clinical setting.

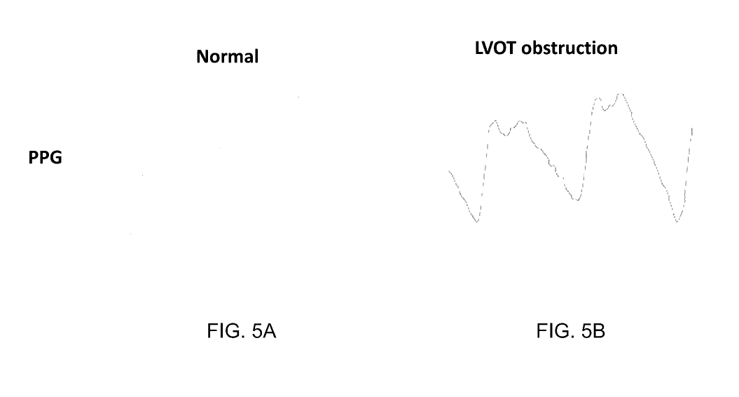

FIGS. 5A and 5B illustrate photoplethysmographic traces from a wristworn sensor from a healthy individual (FIG. 5A) and from an individual with HCM and obstruction (FIG. 5B).

While the systems and methods disclosed herein are largely discussed in terms of HCM with obstruction, they can also be used to diagnose other conditions and hemodynamic abnormalities, such as HCM without obstruction, dilated cardiomyopathy, restrictive cardiomyopathy, valvular heart disease (such as aortic stenosis and aortic regurgitation), heart failure with reduced or preserved ejection fraction, etc.

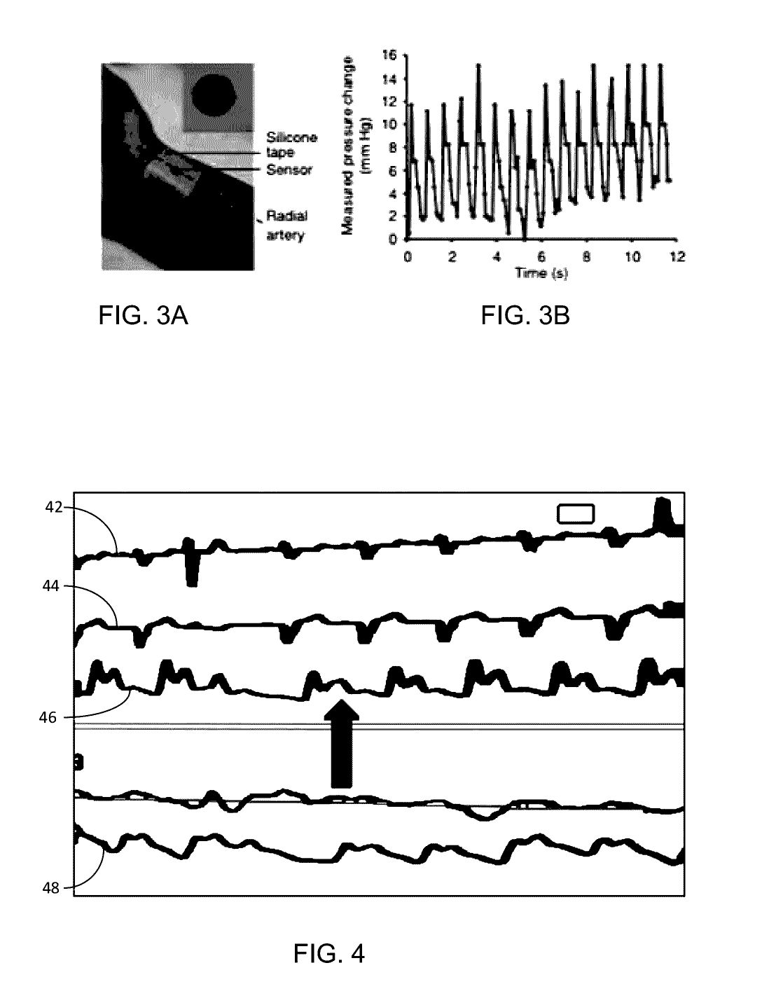

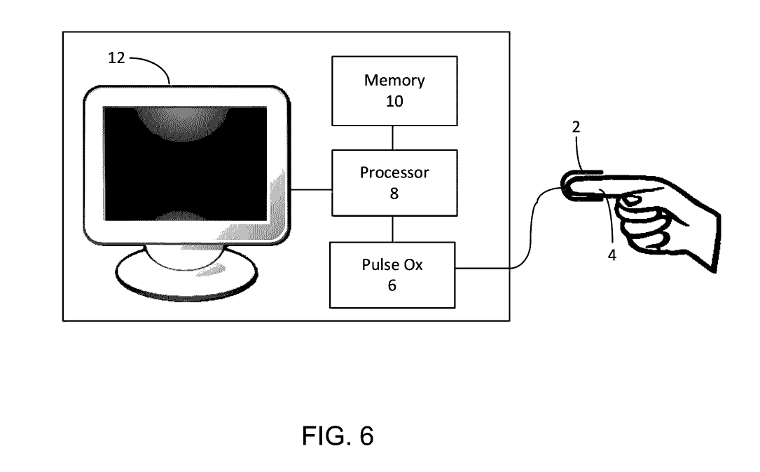

FIG. 6 shows one embodiment of the diagnostic system of this invention. The system obtains a signal from a pulse oximetry sensor 2 attached to the finger 4 of a patient through a pulse oximeter front end 6. A processor 8 obtains plethysmographic information from pulse oximeter front end 6 and generates an arterial blood pressure waveform from that information based on the changes in detected arterial blood over time. In some embodiments, the functionality of pulse oximeter front end 6 may be incorporated into processor 8. While FIG. 6 shows sensor 2 communicating via a wired connection 5, in other embodiments the sensor can communicate with the processor and processor front end wirelessly. In some embodiments, other sensors are used to detect blood flow and/or blood pressure parameters with which a possible LVOT obstruction can be diagnosed. For example, an optical wrist-worn plethysmography sensor as shown in FIG. 3A, and optical sensors, such as those from a digital camera, are also possible. Non-plethysmographic data can also be used (e.g., tonometry).

Processor 8 analyzes the blood pressure waveform to find characteristic shapes or signatures indicative of a cardiac obstruction. Such shapes or signatures may be symptoms of hypertrophic cardiomyopathy. The analysis may include, e.g., analysis of pressure segment slope, uniformity, amplitude, frequency, and/or pulse width. The processor 8 may also compare the waveform generated from plethysmographic information with waveforms stored in memory 10 to identify shapes and/or signatures indicative of cardiac obstruction caused, e.g., by hypertrophic cardiomyopathy. The generated waveform may be identified as an obstruction signature if an obstruction is observed simultaneously via another modality, such as echocardiography, thereby calibrating the device. This embodiment also includes an optional output 12, such as a display, for communicating the generated waveform and/or other results of the analysis to a user.

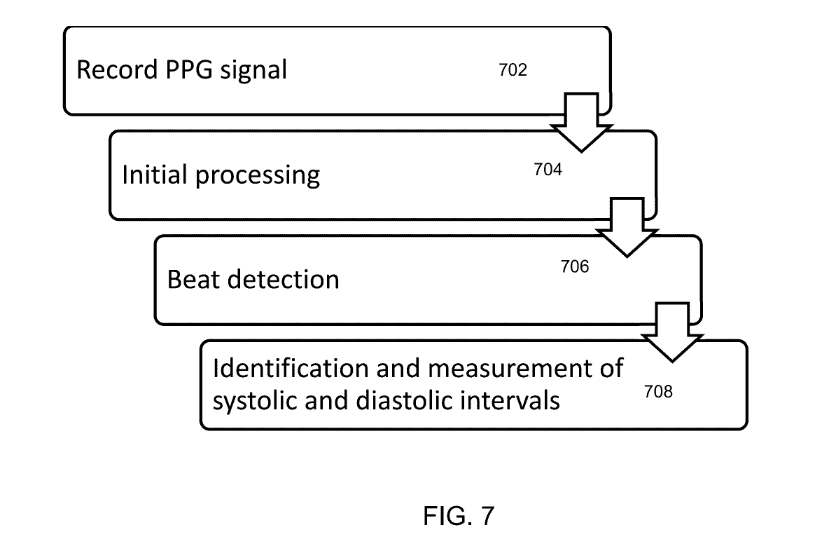

FIG. 7 illustrates an embodiment of a method 700 for identifying LVOT obstruction in a patient. As shown in box 702, the method comprises recording a signal indicative of arterial blood flow (e.g., a PPG signal), for example using an optical wrist or fingertip sensor. The sensor can use green light, at about 520-530 nm or infrared light, at about 850-100 nm. LEDs can be used as the light source. Box 704 shows the initial processing step. This step can include generating a waveform from the signal and detrending the data. The method can further comprise beat detection, shown in box 706. This step involves separating out the waveform into individual beats. The method further comprises identification and measurement of systolic and diastolic intervals, as shown in box 708.

Initial processing can include lowpass filtering, in some embodiments. Lowpass filtering can comprise applying an eighth-order Butterworth lowpass filter (e.g., with a 3 dB point at 18 Hz) and is designed to remove high frequency noise from the PPG-POW signal. Zero-phase filtering can also be implemented, which involves filtering the signal in both forward and backward directions, to eliminate phase distortion.

In some embodiments initial processing comprises detrending or baseline removal. Baseline removal can comprise approximating the baseline of the signal (e.g., by moving averaging with a 2 s window). The baseline component is subtracted from the signal.

Systolic time intervals, for example, left ventricular ejection time (LVET), can be estimated using a beat to beat detection method. The method comprises differentiating the waveform and obtaining the first derivative (d1), the second derivative (d2), the third derivative (d3) and the fourth derivative (d4). Filtering (e.g., moving averaging filtering) can be applied to one or more of the derivatives.

Systolic time intervals, such as LVET, can be estimated from the averaged d3 waveform. There is a close association between d1 and the arterial flow waveform. The end of systolic ejection has been seen to appear as a nadir in the d1 waveform of pulse oximetry waveforms taken at the ear. Based on observations of pulse oximetry waveforms taken at the finger, however, it may be more reliable to identify the end of systolic ejection by detecting a local maximum in the second derivative of d1, corresponding to the diastolic peak in the d3 waveform. As noted above, the derivative waveforms can be averaged. The general form of an average d3 consists of a systolic peak associated with the onset of ejection, one or more peaks produced by wave reflection, and a diastolic peak associated with the end of systolic ejection. The d3 diastolic peak can typically be followed by a sharp falling edge corresponding to a deep trough in d4 and a positive to negative zero cross corresponding to a local maximum in d2. To detect the trough in d4, a trapezium window can be constructed based on the expected physiological range of LVET and multiplied with the negated average d4 waveform. The maximum point within the window is identified as the d4 trough. The negative local minimum in d3 subsequent to the d4 trough would be identified as the d3 trough, whereas the positive to negative zero cross before the d3 trough would correspond to the d2 diastolic peak. The d3 diastolic peak can be selected as the d3 local maximum/inflection point before the d2 diastolic peak, or in some cases, the d3 local maximum/inflection point before the d4 trough if that was much higher than the former. A reference value of LVET can be computed form the average waveform as the time interval between the d3 systolic peak and the d3 diastolic peak.

Beat to beat LVET detection comprises, in some embodiments, identifying the two d3 local maxima from each cardiac period which provided a value of LVET closest to the reference value of LVET. The initial selection of d3 diastolic peak can be based on the assessment of the sign and magnitude of the two selected d3 local maxima and their following d2 local maxima as well as the closeness of their LVET values with the reference LVET, and the final selection can be based on the closeness of their LVET values with the moving average computed from the initial selections. Once the d3 diastolic peak is located, three estimates of LVET can be computed: 1) the first based on the time interval between the d3 systolic peak and the d3 diastolic peak; 2) the second based on the time interval between the d3 systolic peak and the d1 local minimum occurring immediately after the d3 diastolic peak; and 3) the third based on the time interval between the d2 systolic peak and the d2 diastolic peak. The final LVET can be computed as the average of the three estimates unless the d2 diastolic peak was negative or the d1 local minimum occurred much later than the d3 diastolic peak, in which case the final LVET can be obtained solely on the first estimate.

Once, systolic time intervals (e.g., the LVET) are computed, the values can be converted into a numerical pressure gradient between the left ventricle and the aorta. This conversion can be performed by referencing a curve from population data (comparing cath/echo gradient with LVET) or data from that individual (obtained by cath/echo). This pressure gradient describes the degree of left ventricular outflow tract obstruction. Under current clinical guidelines, those patients with a pressure gradient greater than thirty millimeters of mercury are considered to have obstruction and those with a gradient greater than fifty millimeters of mercury may be candidates for septal reduction procedures.

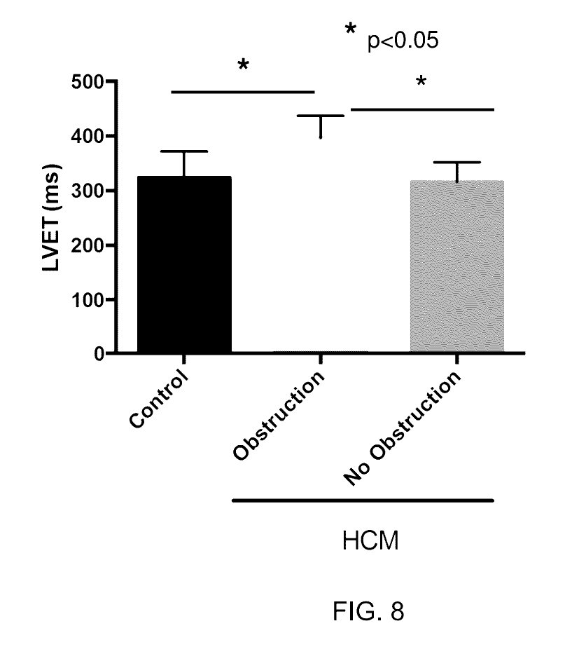

FIG. 8 depicts data from an experimental protocol for determining systolic ejection time from a PPG signal. Twenty patients with a diagnosis of HCM were enrolled in the study. Ten patients had HCM with obstruction and ten patients had HCM without obstruction. Twenty subjects without HCM were studied for comparison. For patients with HCM, a cardiologist determined whether a murmur consistent with LVOT obstruction was present. Five to ten minutes of PPG signal were then recorded at rest. The sensor used was an Amiigo wrist sensor, which used infrared light, with a wavelength about 850-100 nm. The signals were processed as described above, and the LVET was calculated for each patient. T testing was used to determine statistical significance. Generally, the degree of prolongation in LVET found using this protocol agreed with catheter-based measurements of left ventricular outflow tract obstruction, for example, as described in Wigle, E. D., AUGER, P., & MARQUIS, Y. (1967). Muscular Subaortic Stenosis The Direct Relation Between the Intraventricular Pressure Difference and the Left Ventricular Ejection Time. Circulation, 36(1), 36-44, the entirety of which is incorporated by reference herein. The results of the experiment showed that PPG based measurement of LVOT is accurate and can be used to diagnose LVOT.

In some embodiments, the pulse oximeter sensor may be worn for an extended period of time during which the optical information may be stored for later analysis. In other embodiments, the pulse oximeter sensor may be worn for an extended period of time during which the optical information is transmitted wirelessly for generation of an arterial blood pressure waveform and analysis for cardiac obstruction.

In some embodiments, the optical signal may be obtained using, e.g., the light and light detection capabilities of a smartphone or a smart watch. The optical information thus obtained may be analyzed within the smartphone, stored for later analysis or communicated to another device for analysis.

In some embodiments, a blood pressure sensor (such as, e.g., the noninvasive blood pressure sensor shown in FIG. 3 or a blood pressure catheter) may be used to obtain the information from which the arterial blood pressure waveform is generated by the system.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of detecting hypertrophic cardiomyopathy with obstruction in a patient, the method comprising
obtaining a plethysmographic signal from a patient;
generating a waveform from the plethysmographic signal; and
processing and analyzing the waveform to determine whether the patient suffers from hypertrophic cardiomyopathy with obstruction.

2. The method of claim 1, wherein the plethysmographic signal is a pulse oximetry signal.

3. The method of claim 1, wherein the plethysmographic signal is a photoplethysmographic signal.

4. The method of claim 1, wherein obtaining the plethysmographic signal comprises obtaining a signal from a wrist or fingertip sensor.

5. The method of claim 1, wherein processing and analyzing the waveform comprises estimating systolic time intervals from the waveform.

6. The method of claim 1, wherein processing and analyzing the waveform comprises estimating a left ventricular ejection time from the waveform.

7. The method of claim 1, wherein processing and analyzing the waveform comprises determining a pressure gradient between a left ventricle and aorta of the patient.

8. The method of claim 1, wherein processing and analyzing the waveform comprises
estimating a left ventricular ejection time from the waveform; and
converting the left ventricular ejection time into a numerical pressure gradient between a left ventricle and an aorta of the patient.

9. The method of claim 1, wherein obtaining a plethysmographic signal comprises wirelessly obtaining the plethysmographic signal.

10. The method of claim 1, wherein obtaining the plethysmographic signal occurs, at least in part, outside of a clinical setting.

11. The method of claim 1, wherein obtaining the plethysmographic signal from the patient comprises attaching a plethysmographic sensor to the patient.

12. The method of claim 1, further comprising displaying the result of processing and analyzing the waveform.

13. A system for detecting hypertrophic cardiomyopathy with obstruction in a patient, the system comprising
a plethysmographic sensor;
a processor in data communication with the plethysmographic sensor, the processor configured to generate a waveform from the information and to identify hypertrophic cardiomyopathy from the waveform; and
an output mechanism configured to provide to a user the information from the blood sensor, the waveform and/or the indicator.

14. The system of claim 13, wherein the plethysmographic sensor is a pulse oximeter.

15. The system of claim 13, wherein the plethysmographic sensor is a photoplethysmographic sensor.

16. The system of claim 13, wherein the plethysmographic sensor comprises a fingertip or wrist sensor.

17. The system of claim 13, wherein the processor identifying hypertophic cardiomyopathy comprises estimating systolic time intervals from the waveform.

18. The system of claim 13, wherein the processor identifying hypertrophic cardiomyopathy comprises estimating a left ventricular ejection time from the waveform.

19. The system of claim 13, wherein the processor identifying hypertrophic cardiomyopathy comprises determining a pressure gradient between a left ventricle and aorta of the patient.

20. A method for detecting a condition in a patient, the method comprising
obtaining a signal from a sensor indicative of blood flow over time;
generating a waveform from the signal; and
processing and analyzing the waveform to determine whether the patient suffers from the condition.

21. The method of claim 20, wherein the signal is obtained from a photoplethysmographic sensor or a tonometry sensor.

22. The method of claim 20, wherein processing and analyzing the waveform comprises
estimating systolic time intervals from the waveform; and
converting the systolic time intervals into a numerical pressure gradient between a left ventricle and an aorta of the patient.

23. The method of claim 20, wherein processing and analyzing the waveform comprises estimating a left ventricular ejection time from the waveform; and
converting the left ventricular ejection time into a numerical pressure gradient between a left ventricle and an aorta of the patient.

24. The method of claim 20, wherein processing and analyzing the waveform comprises determining a pressure gradient between a left ventricle and aorta of the patient.

25. The method of claim 20, wherein the signal is generated from a wrist-worn or fingertip sensor.

26. The method of claim 20, wherein the condition comprises at least one of HCM with obstruction, HCM without obstruction, dilated cardiomyopathy, restrictive cardiomyopathy, valvular heart disease, and heart failure with reduced or preserved ejection fraction.
